(19) United States
(12) Patent Application Publication
(10) Pub. No.: US 2021/0237937 A1
(43) Pub. Date: Aug. 5, 2021

(54) Compact Reusable Silicone Pouch

(71) Applicant: Junobie, LLC, West Lafayette, IN (US)

(72) Inventor: Nikeytha Monique Ramsey, West Lafayette, IN (US)

(21) Appl. No.: 16/996,862

(22) Filed: Aug. 18, 2020

Related U.S. Application Data
(60) Provisional application No. 62/888,521, filed on Aug. 18, 2019.

(51) Int. Cl. B65D 33/25; B65D 33/00

(52) U.S. Cl. CPC B65D 33/2541; B65D 33/04; B65D 33/004

(57) ABSTRACT

A reusable container having a pouch portion and a slide lock portion. The pouch portion can be a rectangular, flat pouch. The slide lock system can seal a top portion of the pouches sealing mechanism. The sealing mechanism can allow a user to store liquids conveniently therein without risk of spilling while also providing a reusable easy to clean container.

### CROSS-REFERENCE TO RELATED APPLICATION

This U.S. Patent Application claims priority to U.S. Provisional Application: 62/888,521 filed Aug. 18, 2019, the disclosure of which is considered part of the disclosure of this application and is hereby incorporated by reference in its entirety.

### FIELD OF THE INVENTION

This disclosure relates generally to a resealable container. In one aspect, the disclosure relates to planar-shaped, reusable, silicone pouch for storing liquids.

### BACKGROUND

Many mothers collect and store breast milk for feeding to their babies. Such collection, storage and feeding may typically occur in a hospital setting (e.g., in the case of a premature/low birth weigh baby) or in the home environment (e.g., in the case of a healthy baby released from the hospital shortly after birth).

Of note, the collection of such breast milk may typically be carried out using a breast pump (e.g., manual or electric), the storage of such breast milk may typically be carried out using a storage container and the feeding of such breast milk may typically be carried out using a feeding bottle. Currently many mothers have to rely upon a single use plastic container that collects the milk after it is pumped resulting in waste and continuous purchasing of plastic bags.

There exists a need for a reusable, resealable container to store perishable liquids, such as breast milk, that can also be easily washed, reusable, as well as able to withstand a wide range of temperatures as the milk may be frozen and thawed for use.

### BRIEF SUMMARY OF THE INVENTION

Nursing mothers usually store breast milk in bulky glassware or plastic containers. Such containers are not always convenient to carry. The device herein disclosed and described provides a solution to the shortcomings in the prior art through the disclosure of a highly compact, planar pouch for liquids such as breast milk. An object of the invention is to allow nursing mothers to easily store extra containers during periods of nursing. For example, when empty the pouches close flat and can fit easily into small purses and bags.

Another aspect of the disclosure is related to providing a means to easily receive breast milk. An opening of the pouch can be expanded with one hand to receive milk and minimize spillage unlike conventional containers.

Another aspect of the disclosure is related to providing a means to seal the container and prevent leaking quickly. A pouch of the present disclosure can include a slide lock that seals liquids inside the container.

Another aspect of the disclosure is related to providing a means to allow a user to quickly notice the volume of liquid inside the container. A pouch of the present disclosure can further include has a graduated relief on its outside surface.

Another aspect of the present disclosure provides a means to allow pouches to be easily connected or affixed inside of a bag. Each pouch as an eyelet on one corner to receive a ring or clip. A pouch of the present disclosure can also minimize waste by be constructed of reusable silicone that is dishwasher safe and can be reused many times.

In one aspect the present disclosure relates to A reusable container having a pouch portion including a includes a proximate end having an opening and a sealed distal end, a first sidewall and a second sidewall, a bottom sidewall, an interior cavity and sealing mechanism and a slide lock portion configured to seal the sealing mechanism of the pouch portion. In some embodiments, the bottom sidewall can generally concave when in a stored position and configured to allow the pouch to expand the interior cavity when filled with a liquid and retract when empty.

The reusable container can include a sealing mechanism is located on the proximate end having an opening. The reusable container of claim 1 having at least a first channel on the exterior side of the sidewall at the proximate end. The slide lock can have a slot configured to slide along the first channel to apply a compressive force to seal the proximate end of the pouch portion in an engaged position. Additionally, the sidewall of the container can include a graduated volume indicia on a sidewall. Some embodiments, can further include an aperture at corner of the proximate end of the pouch.

The proximate end of the pouch further comprises a sealing mechanism. The sealing mechanism includes a groove member on a first sidewall of the proximate end and a track member on the second side wall of the proximate end, wherein the groove member is configured to interface with the track member when the slide lock is in the engaged position. The sealing mechanism further includes one or more sealing members. The sealing members can generally be perpendicular to the interior sidewall of the proximate end of the pouch. In some embodiments, a first sealing member is located above the groove member or track member. The pouch may also include a second sealing member is located below the groove member or track member. A third sealing member can also be included and located below the second sealing member.

Another aspect of the present disclosure is related to a method of storing liquids, the method including adjusting the opening of the pouch, receiving liquid inside the pouch, and sealing the opening of the pouch. The sealing of the pouch can be accomplished with a the slide lock to compresses the pouch opening creating a seal there between.

The invention now will be described more fully hereinafter with reference to the accompanying drawings, which are intended to be read in conjunction with both this summary, the detailed description and any preferred and/or particular embodiments specifically discussed or otherwise disclosed. This invention may, however, be embodied in many different forms and should not be construed as limited to the embodiments set forth herein; rather, these embodiments are provided by way of illustration only and so that this disclosure will be thorough, complete and will fully convey the full scope of the invention to those skilled in the art.

### BRIEF DESCRIPTION OF THE DRAWINGS

FIG. 1 is an illustration of a perspective view of an exemplary embodiment of a pouch of the present disclosure.

FIG. 2 is an illustration of a perspective view of a user carrying an exemplary embodiment of a pouch of the present disclosure.

FIG. 3 is a front view of an exemplary embodiment of a pouch of the present disclosure having an optional graduated relief scale on its outside surface.

FIG. 4 is a front view of an exemplary embodiment of a pouch of the present disclosure wherein the slide lock is off of the pouch.

FIG. 5 is a back view of an exemplary embodiment of a pouch of the present disclosure wherein the slide lock is off of the pouch.

FIG. 6 is a left side view of an exemplary embodiment of a pouch of the present disclosure wherein the slide lock is off of the pouch.

FIG. 7 is a right-side view of an exemplary embodiment of a pouch of the present disclosure wherein the slide lock is off of the pouch.

FIG. 8 is a perspective view of an exemplary embodiment of a pouch of the present disclosure wherein the slide lock is off of the pouch.

FIG. 9 is a bottom view of an exemplary embodiment of a pouch of the present disclosure.

FIG. 10 is a top view of an exemplary embodiment of a pouch of the present disclosure.

FIG. 11 is a cross-sectional view of an exemplary embodiment of a pouch of the present disclosure along axis BE shown in FIG. 10.

FIG. 12A is a perspective view of a sealing portion of a pouch of the present disclosure.

FIG. 12B is a perspective cross-sectional view of a sealing portion of a pouch of the present disclosure along plane BJ of FIG. 12A.

FIG. 13A is a bottom view of an exemplary embodiment of a slide lock of the present disclosure.

FIG. 13B is a side view of an exemplary embodiment of a slide lock of the present disclosure.

FIG. 13C is a perspective view of an exemplary embodiment of a slide lock of the present disclosure.

### DETAILED DESCRIPTION OF THE INVENTION

The following detailed description includes references to the accompanying drawings, which forms a part of the detailed description. The drawings show, by way of illustration, specific embodiments in which the invention may be practiced. These embodiments, which are also referred to herein as “examples,” are described in enough detail to enable those skilled in the art to practice the invention. The embodiments may be combined, other embodiments may be utilized, or structural, and logical changes may be made without departing from the scope of the present invention. The following detailed description is, therefore, not to be taken in a limiting sense.

Before the present invention of this disclosure is described in such detail, however, it is to be understood that this invention is not limited to particular variations set forth and may, of course, vary. Various changes may be made to the invention described and equivalents may be substituted without departing from the true spirit and scope of the invention. In addition, many modifications may be made to adapt a particular situation, material, composition of matter, process, process act(s) or step(s), to the objective(s), spirit or scope of the present invention. All such modifications are intended to be within the scope of the disclosure made herein.

Unless otherwise indicated, the words and phrases presented in this document have their ordinary meanings to one of skill in the art. Such ordinary meanings can be obtained by reference to their use in the art and by reference to general and scientific dictionaries.

References in the specification to “one embodiment” indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The following explanations of certain terms are meant to be illustrative rather than exhaustive. These terms have their ordinary meanings given by usage in the art and in addition include the following explanations.

As used herein, the term “and/or” refers to any one of the items, any combination of the items, or all of the items with which this term is associated.

As used herein, the singular forms “a,” “an,” and “the” include plural reference unless the context clearly dictates otherwise.

As used herein, the terms “include,” “for example,” “such as,” and the like are used illustratively and are not intended to limit the present invention.

As used herein, the terms “preferred” and “preferably” refer to embodiments of the invention that may afford certain benefits, under certain circumstances. However, other embodiments may also be preferred, under the same or other circumstances.

Furthermore, the recitation of one or more preferred embodiments does not imply that other embodiments are not useful and is not intended to exclude other embodiments from the scope of the invention.

As used herein, the terms “front,” “back,” “rear,” “upper,” “lower,” “right,” and “left” in this description are merely used to identify the various elements as they are oriented in the FIGS, with “front,” “back,” and “rear” being relative to the apparatus. These terms are not meant to limit the elements that they describe, as the various elements may be oriented differently in various applications.

As used herein, the term “coupled” means the joining of two members directly or indirectly to one another. Such joining may be stationary in nature or movable in nature. Such joining may be achieved with the two members or the two members and any additional intermediate members being integrally formed as a single unitary body with one another or with the two members or the two members and any additional intermediate members being attached to one another. Such joining may be permanent in nature or alternatively may be removable or releasable in nature. Similarly, coupled can refer to a two member or elements being in communicatively coupled, wherein the two elements may be electronically, through various means, such as a metallic wire, wireless network, optical fiber, or other medium and methods.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element without departing from the teachings of the disclosure.

In this description, the directional prepositions of up, upwardly, down, downwardly, front, back, top, upper, bottom, lower, left, right and other such terms refer to the device as it is oriented and appears in the drawings and are used for convenience only; they are not intended to be limiting or to imply that the device has to be used or positioned in any particular orientation. Conventional components of the invention are elements that are well-known in the prior art and will not be discussed in detail for this disclosure.

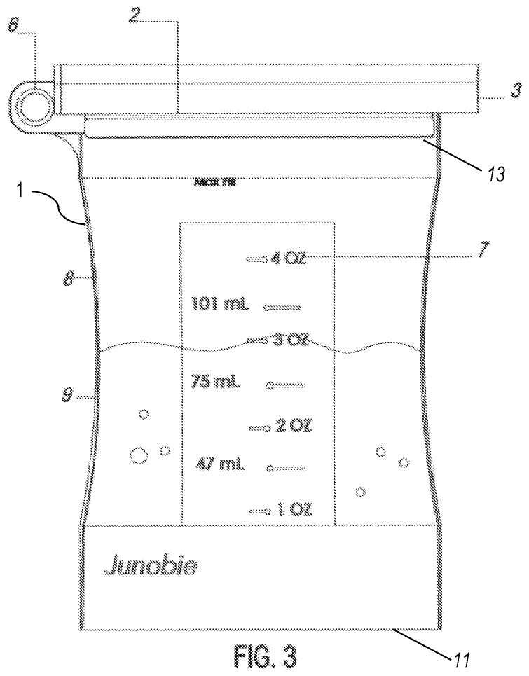

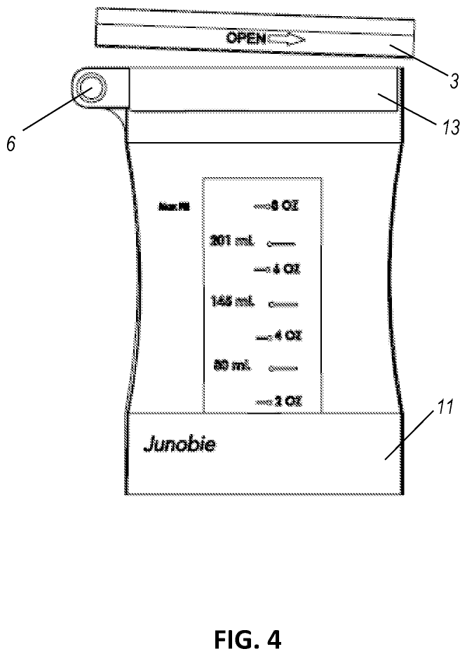

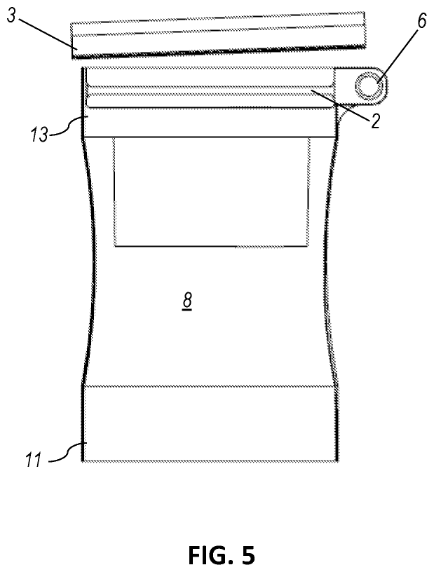

FIG. 1 showing a perspective view of the pouch container 1 of the present disclosure. In some exemplary embodiment, the container 1 can have any suitable shape, such as a generally a rectangular shape and can be made of any suitable material, including but not limited to plastic, polymer, or a flexible, translucent material such as but not limited to silicone, plastic. The container can have a distal end 11 sealed and another proximate end 13 having an opening open. As shown in FIG. 3, some exemplary embodiments the container can further include a slide lock 3 having, which in can have any suitable shape, including but not limited to a rectangular shape. The slide lock 3 can be made of any suitable material including but not limited to a rigid material such as a plastic or polymer.

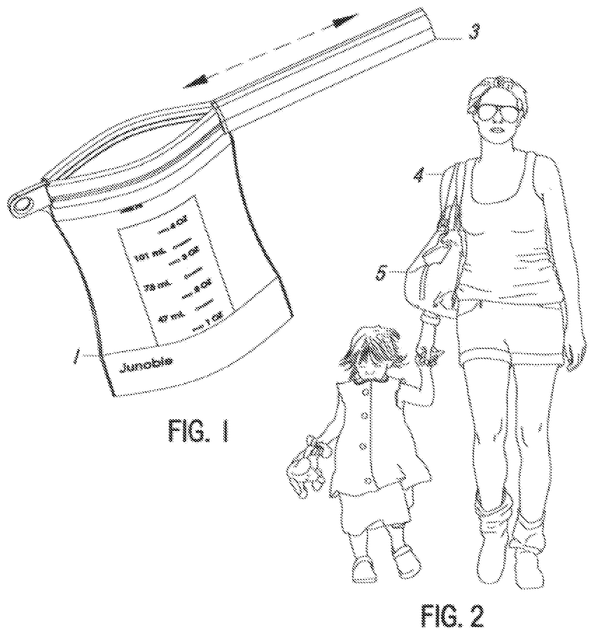

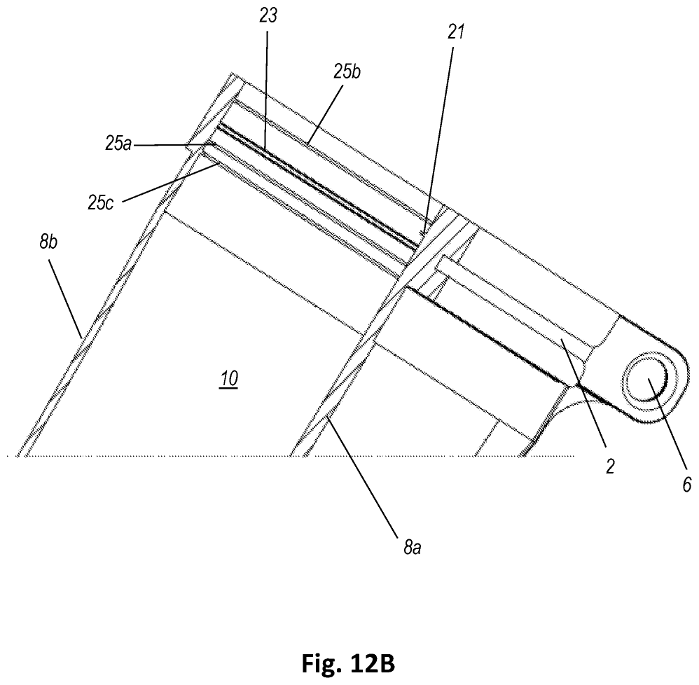

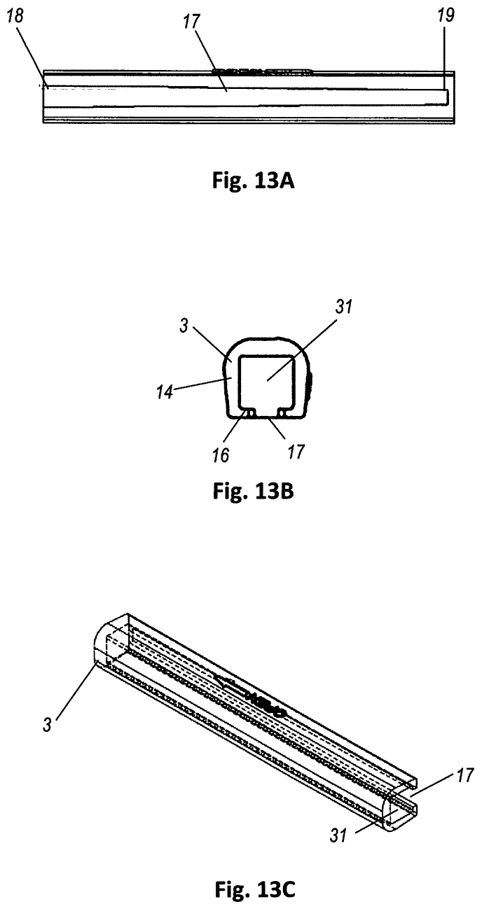

As shown in FIGS. 13A-C, the slide lock 3 can have a slot 17 to provide a compressive force against one or more sealable tracks 21 and corresponding grooves 23 in said pouch that when compressed there between slide lock 3 creating a leak-proof seal therein. The slide lock 3 can be guided on the exterior portion of the proximate end by one or more channels or tracks 2 located on one or both sides of the distal end of the pouch 1. FIG. 2 showing an example of user 4 carrying the invention 1 in carry bag 5.

FIG. 3 illustrates a close-up view of the invention with the invention 1 filled with a liquid 9 having graduated volumetric indicia 7 in a proximal portion of sidewall 8 that includes but is not limited to mL and Oz. etc. The slide lock 3 being slidably installed over pouch tracks or channels 2 at the proximate end 13 having an opening thereby creating a compression seal there between the two sidewalls 8 of the container 1. The figure also showing aperture 6 on a distal corner configured to receive conventional rings and clips therein.

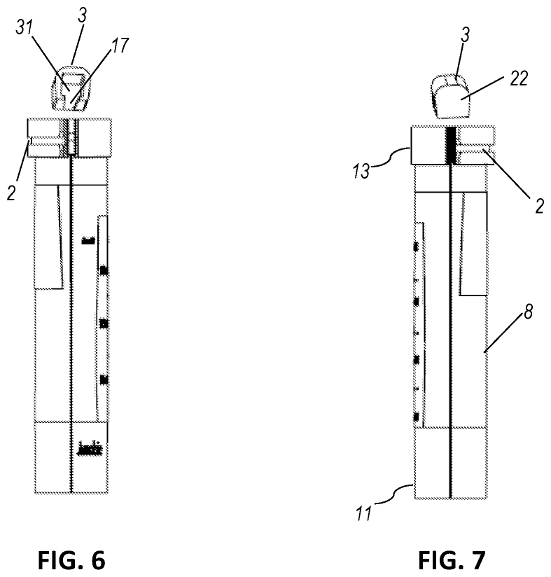

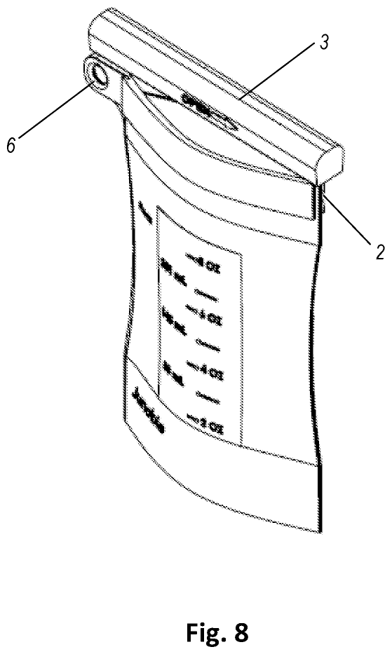

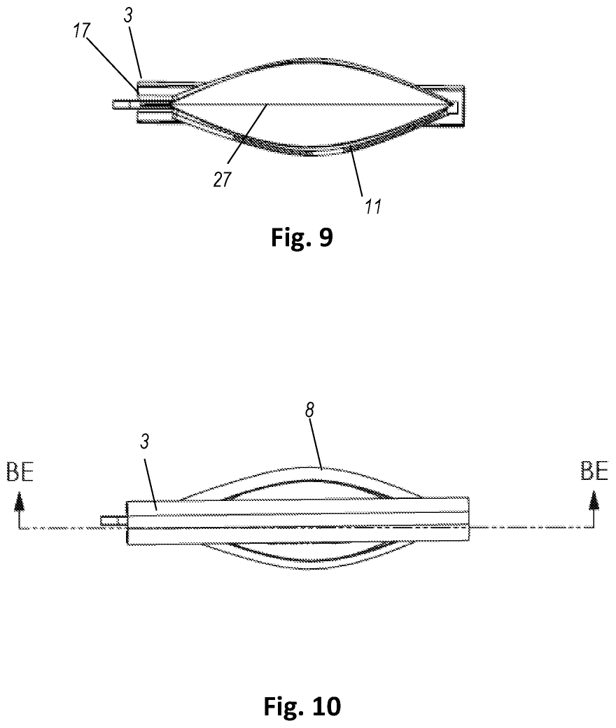

As shown in FIG. 6, the slide lock 2 can have a slot 17 that is configured to allow the top portion of the container 13 to be fluidly sealed and prevent any liquid 9 from escaping the interior portion 10 of the container 1. In some exemplary embodiments, the slot can be configured to have a narrowing configuration that interfaces the channels 2 on the exterior sidewalls 8 of container 1 as shown in FIG. 6 and FIG. 13a. The slide lock can also have a recessed portion or cavity 31 to allow for the top portion 13 of the container to reside inside the cavity 31 of the slide lock 3.

As shown in FIG. 13a, some exemplary embodiments of the slide lock 3 can have a tapered slot 17, wherein the first end 18 of the slot 17 is wider than the second end 19. In some exemplary embodiments, the slot can be formed by one or more overhang portion 16 that extend generally perpendicularly from the sidewall 14 of the slide lock 3. It should be understood that other embodiments can include an opposite taper from the first end to the second end or a uniform slot width across the entire slot. In some exemplary embodiments, the slide lock 3 can have an end wall 22 that can stop the slide lock from sliding off of the top portion 13 of the container 1. The slide lock 3 can be configured to be completely removed from the pouch container 1 to allow a user to completely remove the slide lock 3 from the container 1. This can allow for easier maintenance and cleaning of the container 1 and slide lock 3.

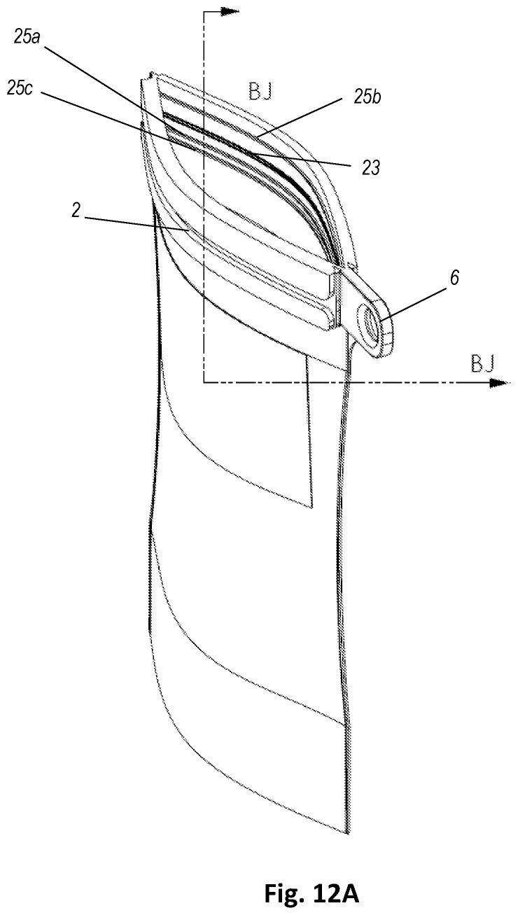

Once the slide lock 3 is coupled to the pouch portion 1 the slot can provide pressure against the two sidewalls 8 of the pouch 1. As shown in FIG. 12, the interior sidewalls of the top portion 13 can include one or more sealing mechanisms. In some exemplary embodiments, the sealing mechanism can include one or more grooves 23 and corresponding tracks 21 to provide a seal to prevent a liquid 9 from spilling out of the interior cavity 10 of the container. In some exemplary embodiments, the sealing mechanism can include two sets of corresponding grooves 23 and tracks 21, wherein once the slide lock is slid from a first position to a second position along the channel 2, the tracks thereby interface with the grooves to create a physical seal. In some exemplary embodiments, the first set of corresponding grooves 23a and tracks 21a can be located above the top edge of the channel 2 and a second set of corresponding grooves 23b and tracks 21b can be located below the bottom edge of the channel. In other embodiments, the all corresponding grooves 23 and tracks 21 can be located above the top edge of the channel 2. As shown in FIG. 12A-B, one exemplary embodiment of the present disclosure a first groove 23a and a first track 21a can b located at the same plane or proximate to the top edge of the channel 2. The sealing mechanism can further include one or more additional sealing members 25 to provide additional sealing and liquid retention within the container. The sealing members can extend perpendicularly from the one or both of the sidewalls of the top portion 13 of the container portion 1.

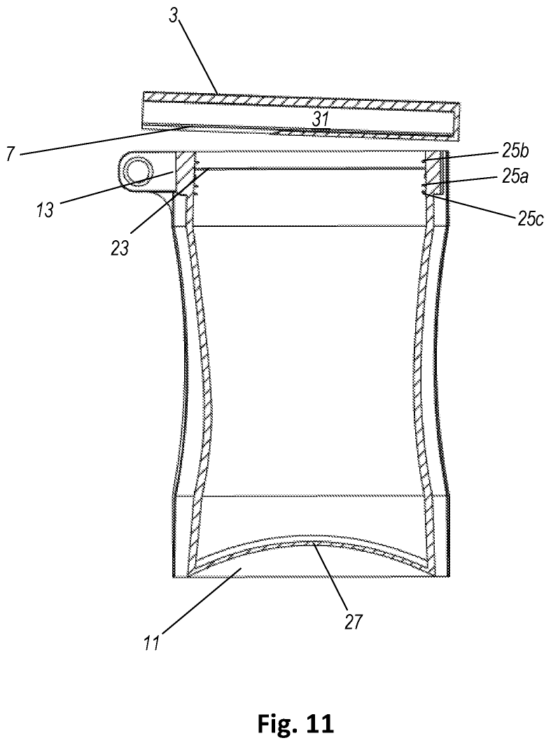

When the slide lock 3 is engaged with the channel 2 on the container 1 and a compressive force is provided to push each side of the sidewall 8 together, the groove 23 and track 21 members can be engaged to form a seal and the sealing members 25a,b,c can be pressed against the opposing sidewall to create an additional barrier to prevent liquid from leaking out of the top portion of the pouch container 1. In some exemplary embodiments the sealing members can be located both above and/or below the groove and track members as shown in FIGS. 11, 12A, and 12B. It should be understood that the sealing members are option and may be located above or below or both of the grooved 23 and track 21 members.

Additionally, in some exemplary embodiments, the bottom wall 27 of the container can be concave in configuration as shown in FIG. 11. The concave configuration can allow for the bag to be easily flattened out for storage after use. When the pouch 1 is filled with a liquid, the bottom wall 27 can be flattened out creating a larger cavity for storage of a liquid within the pouch.

While the invention has been described above in terms of specific embodiments, it is to be understood that the invention is not limited to these disclosed embodiments. Upon reading the teachings of this disclosure many modifications and other embodiments of the invention will come to mind of those skilled in the art to which this invention pertains, and which are intended to be and are covered by both this disclosure and the appended claims. It is indeed intended that the scope of the invention should be determined by proper interpretation and construction of the appended claims and their legal equivalents, as understood by those of skill in the art relying upon the disclosure in this specification and the attached drawings.

What is claimed is:

1. A reusable container comprising:
a pouch portion including a includes a proximate end having an opening and a sealed distal end, a first sidewall and a second sidewall, a bottom sidewall, an interior cavity and sealing mechanism; and
a slide lock portion configured to seal the sealing mechanism of the pouch portion.

2. The reusable container of claim 1, wherein sealing mechanism is located on the proximate end having an opening.

3. The reusable container of claim 1 having at least a first channel on the proximate end.

4. The reusable container of claim 3, wherein the slide lock has a slot configured to slide along the first channel to apply a compressive force to seal the proximate end of the pouch portion in an engaged position.

5. The reusable container of claim 4 having graduated volume indicia on a sidewall.

6. The reusable container of claim 1 having an aperture a corner of the proximate end of the pouch.

7. The reusable container of claim 3, wherein the proximate end of the pouch further comprises a sealing mechanism.

8. The reusable container of claim 7, wherein the sealing mechanism includes a groove member on a first sidewall of the proximate end and a track member on the second side wall of the proximate end, wherein the groove member is configured to interface with the track member when the slide lock is in the engaged position.

9. The reusable container of claim 8, wherein the sealing mechanism further includes one or more sealing members.

10. The reusable container of claim 9, wherein the sealing members are generally perpendicular to the interior sidewall of the proximate end of the pouch.

11. The reusable container of claim 10, wherein a first sealing member is located above the groove member or track member.

12. The reusable container of claim 11, wherein a second sealing member is located below the groove member or track member.

13. The reusable container of claim 12, wherein a third sealing member is located below the second sealing member.

14. The reusable container of claim 10, wherein the bottom wall is generally concave when in a stored position and configured to allow the pouch to expand the interior cavity when filled with a liquid and retract when empty.

15. The reusable container of claim 14, wherein the pouch is comprised of a silicone material.

16. A method of storing liquids, the method comprising:
adjusting the opening of the pouch;
receiving liquid inside the pouch; and
sealing the opening of the pouch.

17. The sealing of the pouch of claim 6 wherein the slide lock of claim 1 compresses the pouch opening creating a seal there between.
